(19) United States
(12) Patent Application Publication
(10) Pub. No.: US 2017/0119793 A1
(43) Pub. Date: May 4, 2017

(54) COMPOSITIONS AND METHODS FOR TREATING BONE AND JOINT INFECTIONS

(71) Applicant: CEM-102 PHARMACEUTICALS, INC., Chapel Hill, NC (US)

(72) Inventor: Prabhavathi FERNANDES, Chapel Hill, NC (US)

(21) Appl. No.: 15/317,578

(22) PCT Filed: Jun. 12, 2015
(86) PCT No.: PCT/US15/35596

§ 371 Date: Dec. 9, 2016

Related U.S. Application Data
(60) Provisional application No. 62/011,215, filed on Jun. 12, 2014.

(51) Int. Cl. A61K 31/575; A61K 38/14; A61K 31/546; A61K 31/5377; A61K 31/422; A61K 31/43; A61K 45/06; A61K 38/12

(52) U.S. Cl. CPC A61K 31/575; A61K 45/06; A61K 38/14; A61K 38/12; A61K 31/5377; A61K 31/422; A61K 31/43; A61K 31/546

(57) ABSTRACT

The invention described herein pertains to the treatment of infections arising in bones and/or joints. The invention described herein also pertains to the treatment of infections arising from implants and prostheses. Bone and joint infections (BJIs) represent a serious medical condition that, if untreated, can lead to complete loss or loss of use of bones or, joints in the patient, and even death. BJIs include many diseases, such as acute and chronic osteomyelitis, including pediatric osteomyelitis, septic arthritis, and others.

### CROSS-REFERENCE TO RELATED APPLICATIONS

This application claims the benefit under 35 U.S.C. §119(e) of U.S. Provisional Application Ser. No. 62/011,215, filed Jun. 12, 2014, the disclosure of which is incorporated herein by reference.

### TECHNICAL FIELD

The invention described herein pertains to the treatment of infections arising in bones and/or joints. The invention described herein also pertains to the treatment of infections arising from implants and prostheses.

### BACKGROUND AND SUMMARY OF THE INVENTION

Bone and joint infections (BJIs) represent a serious medical condition that, if untreated, can lead to complete loss or loss of use of bones or joints in the patient, and even death. BJIs include many diseases, such as acute and chronic osteomyelitis, including pediatric osteomyelitis, septic arthritis, and others.

BJIs also include infections accompanying bone and joint replacement procedures. In particular, prosthetic joint infections (PHs) may arise during and/or after medical procedures where the prostheses are implanted in the host animal or patient. Such infections are difficult to treat and generally require longer courses of treatment than typical infections, including up to and greater than a year following the implantation. Joint replacement surgery has become increasingly common and with it infection of the implanted joint hardware and nearby bone and soft tissue. Approximately 1,000,000 hip and knee prostheses were projected to be implanted in the US in 2012; given an estimated 1 to 2% infection rate, up to 20,000 patients/year may develop prosthetic joint infection (PJI).

There are three typical approaches to treat PJI, the most common approach being the 2-stage exchange procedure. The 2-stage exchange procedure involves the following steps: removal of the infected prosthesis with debridement of infected bone and soft tissue; placement of a polymethacrylate (or other appropriate material) spacer designed to preserve the joint space and, if articulated, to maintain mobility (spacers are typically impregnated with antibiotics); intravenous (IV) antibiotic administration for approximately 6 weeks; a period of time off antibiotics; then removal of the spacer with implantation of a new prosthetic joint. Many prosthetic joint infections in the US are treated in this manner. Alternatively, a 1-stage joint replacement procedure can be used in selected cases of PJI, where the risks of repeated surgery are deemed to outweigh the potential benefit of prolonged antibiotic therapy prior to implantation of the new device. A third approach involves debridement and prosthesis retention, the DAR strategy.

Subjects with bone or joint infections (with or without prosthetic material), who are unable to undergo definitive surgery to remove infected bone, sequestrations, or foreign material because of poor health status, age, or comorbidities, generally undergo an initial course of IV antibiotics followed by more limited surgery, consisting of debridement and irrigation. Often, chronic suppressive antibiotic therapy is utilized thereafter.

Some subjects experience an infection relapse, as manifested by bacteremia, purulent discharge, wound dehiscence or pain, at some point following cessation of antibiotic therapy. Often, these subjects need to be hospitalized to receive another cycle of debridement, irrigation, and antibiotics (IV followed by oral antibiotic therapy for 3-6 months or longer). In some cases, the suppressive oral antibiotic therapy is not able to eliminate or reduce the pain and/or the wound drainage, or to improve the ability to ambulate due to decreased weight bearing or lack of joint function. In addition, dosing or duration of currently available oral therapies may be limited by the emergence of treatment-limiting or treatment-related adverse events (AE) (such as renal toxicity, allergy, bone marrow suppression, paresthesias, or skin discoloration), intolerance, toxicity, comorbidities (such as renal insufficiency), or emergence of antimicrobial resistance. When any of these events happen, the oral antibiotic must be replaced.

Every time the infection relapses, the subject may be hospitalized to undergo another cycle of irrigation, debridement and antibiotic therapy (IV followed by oral antibiotic therapy). In some cases, amputation of the infected limb may be considered as the last resort.

There is a continuing need in the US for antibacterial therapies to treat BJIs and PJIs, and in particular, a need for therapies that do not require long-term IV drug administration, and which are effective against the majority of gram-positive pathogens associated with these infections, including methicillin-resistant S. aureus (MRSA), methicillin-sensitive S. aureus (MSSA), and coagulasenegative staphylococci (CoNS). Without being bound by theory, it is believed herein that therapies that do not require long-term IV drug administration, and instead rely on oral administration options will lead to better patience compliance and better outcomes.

Fusidic acid (FA) is a tetracyclic triterpenoid or fusidane (steroidal) antibiotic derived from the fungus Fusidium coccineum that inhibits bacterial protein synthesis. FA is effective against gram-positive bacteria such as Staphylococcus species and Corynebacterium species (L. Verbist, J. Antimicro. Chemo. 25, Suppl. B, 1-5 (1990); A. Bryskier, Fusidic Acid, Chapter 23, in Antimicrobial Agents: Antibacterials and Antifungals (Andre Bryskier, Ed., ASM Press, Washington, USA, 2005)). FA is also active against Group A beta-hemolytic streptococci, or Streptococcus pyogenes (L. Verbist, J. Antimicro. Chemo. 25, Suppl. B, 1-5 (1990); A. Bryskier, Fusidic Acid, Chapter 23, in Antimicrobial Agents: Antibacterials and Antifungals (Andre Bryskier, Ed., ASM Press, Washington, USA, 2005); Skov et al., Diag. Micro. Infect. Dis. 40:111-116 (2001)). FA is also active against 99.5 to 99.7% of Enterococcus spp. isolates with an MIC ≦8 μg/mL globally.

FA was developed for clinical use in the 1960s and it is approved for human use outside of the United States, such as in the UK, Canada, Europe, Israel, Australia and New Zealand. It is typically prescribed at doses of 500 mg TID for treating skin and skin structure infections caused by Staphylococcus aureus (A. Bryskier, Fusidic Acid, Chapter 23, in Antimicrobial Agents: Antibacterials and Antifungals (Andre Bryskier, Ed., ASM Press, Washington, USA, 2005); Collignon et al., Int'l J. Antimicrobial Agents 12:S45-S58 (1999); D. Spelman, Int'l J. Antimicrobial Agents 12:S59-S66 (1999)), although some physicians have routinely prescribed the compound at 500 mg BID for treating skin and skin structure infections due to the long half-life of the compound (Fusidic Acid, in Principles and Practice of Infectious Diseases, 6th ed. (Mandell et al. eds., Elsevier, 2006)).

FA is well absorbed orally, with around 98% oral bioavailability. The compound is also well distributed. The pharmacokinetics of FA are known to be non-linear (D. Reeves, J. Antimicrob. Chemo. 20:467-476 (1987); J. Turnidge, Int'l J. Antimicrobial Agents 12:S23-S34 (1999); Vaillant et al., Br. J. Dermatol. 126:591-595 (1992); Guttler et al., Br. J. Pharmacol. 43(1):151-160 (1971); Taburet et al., J. Antimicrob. Chemo. 25, Supp. B:23-31 (1990)). Average peak blood concentrations of 22 to 33 ug/mL are reached 2 to 3 hours following single 500 mg oral doses of the tablets (Vaillant et al., Br J Dermatol. 126(6):591-595 (1992)). The Cmax in plasma starts at around 30 ug/ml after the first day of treatment (500 mg TID) and climbs to 140 ug/ml at steady state, which is achieved after the 4th day of a seven to fourteen day course of therapy. The plasma half-life of FA has been found to be between 10 and 12 hours, resulting in significant accumulation with repeated doses.

Based on routine use, the susceptibility breakpoint criteria have been calculated for S. aureus to be: susceptible=MICs <2 micrograms per ml (MIC90=0.12 ug/ml), zone diameters of >21 mm (L. Verbist, J. Antimicro. Chemo. 25, Suppl. B, 1-5 (1990); Skov et al., Diag. Micro. Infect. Dis. 40:111-116 (2001)). Based on these criteria, Group A beta hemolytic Streptococcus have been classified as moderately susceptible: MICs >4-16 micrograms per ml (MIC90 4-8 ug/ml) (Skov et al., Diag. Micro. Infect. Dis. 40:111-116 (2001); Collignon et al., Int'l J. Antimicrobial Agents 12:S45-S58 (1999); Coutant et al., Diagn Microbiol Infect Dis 25:9-13 (1996)).

Protein binding of FA in the blood is substantial, approximately 95-97% (K. Christiansen, International Journal of Antimicrobial Agents 12:S3-S9 (1999); Coutant et al., Diagn Microbiol Infect Dis 25:9-13 (1996); D. Reeves, J. Antimicrob. Chemo. 20:467-476 (1987); J. Turnidge, Int'l J. Antimicrobial Agents 12:S23-S34 (1999); Rieutord et al., Int'l J. Pharmaceutics 119:57-64 (1995)). Therefore, the true MIC is much higher in vivo based upon bioavailable drug (D. Reeves, J. Antimicrob. Chemo. 20:467-476 (1987); J. Turnidge, Int'l J. Antimicrobial Agents 12:S23-S34 (1999)). For Staphylococcus, the MIC is increased by 8-10 fold, i.e., to about 2.5 micrograms per ml or higher in plasma protein. Although the MIC is raised, the drug level is still effective for activity against Staphylococcus as trough blood levels of 15-30 micrograms are obtained with standard dosing (500 mg TID).

FA has been shown to penetrate into avascular foci of infection, such as bone sequestra, in subjects with osteomyelitis. Sodium fusidate has been found in synovial fluid in subjects with rheumatoid arthritis and osteoarthritis, in burn crusts, in pus and in aqueous humor of uninflamed eyes.

It has been reported that the low multiples of the MIC observed under standard dosing may result in resistance selection, For example, after the first day of dosing, when the exposure is to low concentrations of FA, S. aureus resistant mutants can be selected. Once resistance has developed, FA is not effective against the resistant strains at the high blood levels of FA that can be achieved after multiple days of standard dosing as the resistant S. aureus strains can have MICs >256 ug/ml. Resistance is reported to occur if FA is used as a single drug as the resistance frequency at 4 and 8 times the MIC is in the range of 10-6 or 10-8 (Evans et al., J. Clin. Path. 19:555-560 (1966); Hansson et al., J. Mol. Biol. 348:939-949 (2005), Jensen et al., Acta Pathol Microbiol Scand. 60:271-284 (1964); Besier et al., Antimicrob. Agents Chemo., 49(4):1426-1431 (2005); Gemmell et al., J. Antimicrobial Chemo. 57:589-608 (2006)). It has also been published that at 15 to 30 micrograms of free drug, the resistance frequency is <10-11 or 10-13, respectively (Gemmell et al., J. Antimicrobial Chemo. 57:589-608 (2006)). Although blood levels can be high, free FA concentrations do not achieve these high concentrations due to the substantial protein binding of FA. Also, when administered with food, the Cmax could be as much as 25% less than without food. Although the actual MIC is <0.25 ug/ml for S. aureus, with protein binding at 96-98%, at a Cmax of 40 ug/ml, only 1.2 ug/ml would be bioavailable. Further, steady state high levels of FA are not reached until after the 4th day with the standard dosing regimen of 500 mg TID (Saggers et al., Brit. J. Clin. Prac. 22(10):429-430 (1968); J. Turnidge, Int'l J. Antimicrobial Agents 12:S23-S34 (1999)). The amount of drug administered to a patient daily cannot simply be increased. As mentioned above, repeated high level drug dosing results in nausea and vomiting in some patients (K. Christiansen, International Journal of Antimicrobial Agents 12:S3-S9 (1999); Nordin et al., Eur. J. Clin. Res. 5:97-106 (1994)). Co-administration with rifampin is one means to avoid resistance in S. aureus and monotherapy is not recommended for treating infections (Howden et al., Clin. Infec. Dis. 42:394-400 (2006); Gould et al., J. Antimicrobial Chemo. 63:849-861 (2009)).

Fusidic acid (FA, CEM-102), the only member of the fusidane class of antibiotics, inhibits bacterial protein synthesis through binding to and inhibition of elongation factor G (EF-G). It has potent activity against the majority of gram-positive pathogens associated with PJIs, including staphylococci, including methicillin resistant Staphylococcus aureus (MRSA), methicillin susceptible Staphylococcus aureus (MSSA), and coagulase-negative staphylococci (CoNS), Corynebacterium, Propionibacterium acnes, Clostridium, Enterococcus and Streptococcus spp.

However, the successful treatment of bone and joint infections (BJIs), including but not limited to acute and chronic osteomyelitis, pediatric osteomyelitis, vertebral infection, septic arthritis, and prosthetic and other device related infections (PJIs) using FA depends at least in part the ability to achieve therapeutically effective concentrations of FA in the joint and/or bone tissue.

Because BJIs are often treated for extended periods of time, typically several months to as high as several years, the successful treatment of BJIs using FA also depends at least in part the ability to decrease the potential for resistance development. Rifampin (RIF) has been used often as a companion antibacterial agent with FA for that purpose. Moreover, RIF initially appears as a very attractive candidate for treating BJIs due to its ability to penetrate and disperse biofilms, which often accompany BJIs and especially PJIs.

Therapeutic approaches to treating PJIs include a renewed focus on the bacterial biofilm environment on the prosthetic material. Bacterial biofilms reduce treatment efficacy through a variety of mechanisms, including poor antibiotic penetration, protection from host immune response effector cells, and reduced metabolic activity of resident bacteria that may enter a stationary growth phase. Use of bone and biofilm penetrating antibiotics, aggressive debridement to remove biofilm, and removal of biofilm contaminated prosthetic material have all become integral aspects of PJI therapy.

However, RIF has been unexpectedly discovered herein to exhibit a drug-drug interaction (DDI) with FA. In particular, it has been discoverer herein that co-administration of RIF with FA decreases the therapeutic efficacy of FA. It has been discovered herein that RIF induces certain metabolic processes in the host animal leading to increased metabolism of FA, lessening the efficacy of the FA.

Accordingly, a need exists for new methods for treating BJIs that will provide both therapeutically effective concentrations of FA at the site of infection, and not be prone to high resistance development from prolonged therapy.

Described herein are methods for treating BJIs using FA co-administered with a second agent other than RIF. Illustrative second agents include lipopeptide antibiotics, glycopeptide antibiotics, lipoglycopeptide antibiotics, oxazolidinone antibiotics, and beta-lactam antibiotics.

Also described herein are methods for treating BJIs using FA co-administered with a second agent other than RIF, and a third agent. Illustrative third agents include compounds that are active against Gram-negative bacteria and/or anaerobic bacteria infections, such as but not limited to aztreonam, metronidazole, and the like.

Illustratively, the dose of the second and/or third agent is the conventional approved dose for monotherapy using each second and/or third agent.

It is to be understood that the doses are adjusted for patient body weight, age and other comorbidities, or other patient specific criteria, including but not limited to renal clearance, hepatic insufficiency, and cardiac comorbidities.

Without being bound by theory, it is believed herein that when treatment using the compounds, kits, and methods described herein is started, there is potentially already present a large bacterial load at the site of infection. It is further believed that a rapid decrease in the bacterial load using the compounds, kits, and methods described herein will decrease the opportunity or resistance to develop. The components and dosing protocols included in the kits and methods described herein are illustratively adapted for rapidly decreasing the bacterial load.

### BRIEF DESCRIPTION OF THE DRAWING

FIG. 1 shows the FA plasma concentrations over time in six individual human host animals.

### DETAILED DESCRIPTION

It has been discovered that a combination of a lipopeptide, glycopeptide or lipoglycopeptide antibiotic and FA or a pharmaceutically acceptable salt thereof is useful in treating bone and joint infections. Without being bound by theory, it is believed herein that cotherapy with RIF is not required to manage resistance development when using the methods and kits described herein.

It has also been discovered that a combination of an oxazolidinone antibiotic and FA or a pharmaceutically acceptable salt thereof is useful in treating bone and joint infections.

It has also been discovered that a combination of a beta-lactam antibiotic and FA or a pharmaceutically acceptable salt thereof is useful in treating bone and joint infections.

Illustrative lipopeptide, glycopeptide and lipoglycopeptide antibiotics include, but are not limited to oritavancin, daptomycin, dalbavancin, vancomycin, teicoplanin, telavancin, ramoplanin, decaplanin, and the like, and pharmaceutically acceptable salts of any of the foregoing.

Illustrative oxazolidinone antibiotics include, but are not limited to linezolid, tedizolid, and the like, and pharmaceutically acceptable salts of any of the foregoing.

Illustrative beta-lactam antibiotics include, but are not limited to nafcillin, cefazolin, ceftriaxone, cephalexin, dicloxacillin, and the like, and pharmaceutically acceptable salts of any of the foregoing.

Illustrative other antibiotics include, but are not limited to co-trimoxazole (trimethoprim 160 mg+sulfamethoxazole 800 mg q8 h), minocycline, and the like, and pharmaceutically acceptable salts of any of the foregoing.

Several illustrative embodiments of the invention are described by the following clauses:

A method for treating a bone or joint infection (BJI), the method comprising administering FA and administering one or more agents selected from the group consisting of lipopeptide antibiotics, glycopeptides antibiotics, lipoglycopeptide antibiotics, oxazolidinone antibiotics, beta-lactam antibiotics, and combinations thereof.

A kit or package comprising a dose, including a unit dose or unit dosage form, of FA; and a dose, including a unit dose or unit dosage form, of one or more agents selected from the group consisting of lipopeptide antibiotics, glycopeptides antibiotics, lipoglycopeptide antibiotics, oxazolidinone antibiotics, beta-lactam antibiotics, and combinations thereof, and a set of instructions for using the dose of FA and each dose of the one or more agents.

The method or kit of the foregoing clauses wherein the dose of FA is present in therapeutically effective amount to treat a bone or joint infection (BJI).

The method or kit of any of the foregoing clauses wherein each dose of the one or more agents is present in therapeutically effective amount to treat a bone or joint infection (BJI).

The method or kit of any of the foregoing clauses wherein the combination of the dose of FA and the dose of the one or more agents is present in therapeutically effective amount to treat a bone or joint infection (BJI).

The method or kit of any of the foregoing clauses wherein the BJI is a prosthetic joint infection (PJI).

The method or kit of any of the foregoing clauses wherein the BJI is hip PJI, or prosthetic knee infection, or a spacer infection.

The method or kit of any of the foregoing clauses wherein the first administration of FA and the administration of at least one of the one or more agents is performed on the same day.

The method or kit of any of the foregoing clauses wherein the first administration of FA and the administration of at least one of the one or more agents is performed on the first day.

The method or kit of any of the foregoing clauses wherein the administration of at least one of the one or more agents is performed on a day at least one day before the first administration of FA.

The method or kit of any of the foregoing clauses further comprising the step of discontinuing the administration of the one or more agents after a predetermined period of time, and continuing the administration of FA.

The kit of any of the foregoing clauses wherein the set of instructions describes a dosing protocol described herein, or according to a method described herein.

The method or kit of any of the foregoing clauses wherein the FA is administered at a dose of at least 1200 mg/day.

The method or kit of any of the foregoing clauses wherein the FA is administered at a dose of at least 1800 mg/day.

The method or kit of any of the foregoing clauses wherein the FA is administered on one day at a dose of at least 3000 mg/day.

The method or kit of any of the foregoing clauses wherein the FA is administered on subsequent days at a dose of at least 1200 mg/day.

The method or kit of any of the foregoing clauses wherein the FA is administered on subsequent days at a dose of at least 1800 mg/day.

The method or kit of any of the foregoing clauses wherein the dose is b.i.d., such as 1500 mg b.i.d. for a total daily dose (TDD) of 3000 mg, or 900 mg b.i.d. for a TDD of 1800 mg.

The method or kit of any of the foregoing clauses wherein the dose is b.i.d., such as 1500 mg b.i.d. for a total daily dose (TDD) of 3000 mg, or 600 mg b.i.d. for a TDD of 1200 mg.

The method or kit of any of the foregoing clauses wherein at least one agent is a lipopeptide antibiotic.

The method or kit of any of the foregoing clauses wherein at least one agent is a glycopeptide antibiotic.

The method or kit of any of the foregoing clauses wherein at least one agent is a lipoglycopeptide antibiotic.

The method or kit of any of the foregoing clauses wherein at least one agent is an oxazolidinone antibiotic.

The method or kit of any of the foregoing clauses wherein at least one agent is an beta-lactam antibiotic.

The method or kit of any of the foregoing clauses wherein the second or third agent does not induce Cytochrome P450 3A4 (CYP3A4).

The method or kit of any of the foregoing clauses wherein the agent is oritavancin or a salt thereof.

The method or kit of any of the foregoing clauses wherein the agent is oritavancin or a salt thereof intravenously administered at a dose of about 1200 mg q.s.

The method or kit of any of the foregoing clauses wherein the agent is oritavancin or a salt thereof administered once, then discontinued for a predetermined period of time.

The method or kit of any of the foregoing clauses wherein the agent is administered semi-monthly or monthly.

The method or kit of any of the foregoing clauses wherein the agent is administered once.

The method or kit of any of the foregoing clauses wherein the agent is administered twice.

The method or kit of any of the foregoing clauses wherein the agent is dalbavancin or a salt thereof.

The method or kit of any of the foregoing clauses wherein the agent is dalbavancin or a salt thereof intravenously administered at a dose of 1000 mg once per week, or 500 mg once per week.

The method or kit of any of the foregoing clauses wherein the agent is dalbavancin or a salt thereof administered weekly.

thereof intravenously administered at a dose of 1000 mg once per week, or 1000 mg in the first week, and 500 mg in the second and subsequent weeks.

The method or kit of any of the foregoing clauses wherein the agent is dalbavancin or a salt thereof administered for at least 1 week.

The method or kit of any of the foregoing clauses wherein the agent is dalbavancin or a salt thereof administered for at least 2 weeks.

The method or kit of any of the foregoing clauses wherein the agent is dalbavancin or a salt thereof, and the administering thereof is discontinued for a predetermined period of time.

The method or kit of any of the foregoing clauses wherein the agent is dalbavancin or a salt thereof administered for 2 weeks.

The method or kit of any of the foregoing clauses wherein the agent is vancomycin or a salt thereof.

The method or kit of any of the foregoing clauses wherein the agent is vancomycin or a salt thereof intravenously administered at a dose of 15 mg/kg q12 h.

The method or kit of any of the foregoing clauses wherein the agent is vancomycin or a salt thereof administered daily.

The method or kit of any of the foregoing clauses wherein the agent is vancomycin or a salt thereof administered twice daily.

The method or kit of any of the foregoing clauses wherein the agent is vancomycin or a salt thereof administered for at least 1 week.

The method or kit of any of the foregoing clauses wherein the agent is vancomycin or a salt thereof administered for at least 2 weeks.

The method or kit of any of the foregoing clauses wherein the agent is vancomycin or a salt thereof, and the administering thereof is discontinued for a predetermined period of time.

The method or kit of any of the foregoing clauses wherein the agent is vancomycin or a salt thereof is administered for 1-2 weeks, then discontinued for a predetermined period of time.

The method or kit of any of the foregoing clauses wherein the agent is vancomycin or a salt thereof administered for 1 week.

The method or kit of any of the foregoing clauses wherein the agent is vancomycin or a salt thereof administered for 2 weeks.

The method or kit of any of the foregoing clauses wherein the agent is daptomycin or a salt thereof.

The method or kit of any of the foregoing clauses wherein the agent is daptomycin or a salt thereof intravenously administered at a dose of 6 mg/kg q24 h.

The method or kit of any of the foregoing clauses wherein the agent is daptomycin or a salt thereof administered daily.

The method or kit of any of the foregoing clauses wherein the agent is daptomycin or a salt thereof administered for at least 1 week.

The method or kit of any of the foregoing clauses wherein the agent is daptomycin or a salt thereof administered for at least 2 weeks.

The method or kit of any of the foregoing clauses wherein the agent is daptomycin or a salt thereof, and the administering thereof is discontinued for a predetermined period of time.

The method or kit of any of the foregoing clauses wherein the agent is daptomycin or a salt thereof administered for 1 week.

The method or kit of any of the foregoing clauses wherein the agent is daptomycin or a salt thereof administered for 2 weeks.

The method or kit of any of the foregoing clauses wherein the agent is telavancin or a salt thereof.

The method or kit of any of the foregoing clauses wherein the agent is telavancin or a salt thereof intravenously administered at a daily dose of 10 mg/kg.

The method or kit of any of the foregoing clauses wherein the agent is linezolid or a salt thereof.

The method or kit of any of the foregoing clauses wherein the agent is linezolid or a salt thereof intravenously or orally administered at a dose 600 mg q12 h.

The method or kit of any of the foregoing clauses wherein the agent is linezolid or a salt thereof administered daily.

The method or kit of any of the foregoing clauses wherein the agent is linezolid or a salt thereof administered twice daily.

The method or kit of any of the foregoing clauses wherein the agent is linezolid or a salt thereof administered for at least 1 week.

The method or kit of any of the foregoing clauses wherein the agent is linezolid or a salt thereof administered for at least 2 weeks.

The method or kit of any of the foregoing clauses wherein the agent is linezolid or a salt thereof, and the administering thereof is discontinued for a predetermined period of time.

The method or kit of any of the foregoing clauses wherein the agent is linezolid or a salt thereof administered for 1 week.

The method or kit of any of the foregoing clauses wherein the agent is linezolid or a salt thereof administered for 10 days.

The method or kit of any of the foregoing clauses wherein the agent is linezolid or a salt thereof administered for 2 weeks.

The method or kit of any of the foregoing clauses wherein the agent is tedizolid or a salt thereof.

The method or kit of any of the foregoing clauses wherein the agent is tedizolid or a salt thereof orally administered at a daily dose of 200 mg.

The method or kit of any of the foregoing clauses wherein the agent is nafcillin or a salt thereof.

The method or kit of any of the foregoing clauses wherein the agent is nafcillin or a salt thereof intravenously administered at a dose of 1.5-2 g q4-6 h.

The method or kit of any of the foregoing clauses wherein the agent is nafcillin or a salt thereof administered for at least 1 week.

The method or kit of any of the foregoing clauses wherein the agent is nafcillin or a salt thereof administered for at least 2 weeks.

The method or kit of any of the foregoing clauses wherein the agent is nafcillin or a salt thereof, and the administering thereof is discontinued for a predetermined period of time.

The method or kit of any of the foregoing clauses wherein the agent is nafcillin or a salt thereof administered for 1 week.

The method or kit of any of the foregoing clauses wherein the agent is nafcillin or a salt thereof administered for 2 weeks.

The method or kit of any of the foregoing clauses wherein the agent is cefazolin or a salt thereof.

The method or kit of any of the foregoing clauses wherein the agent is cefazolin or a salt thereof intravenously administered at a dose of 1-2 g q8 h.

The method or kit of any of the foregoing clauses wherein the agent is cefazolin or a salt thereof administered for at least 1 week.

The method or kit of any of the foregoing clauses wherein the agent is cefazolin or a salt thereof administered for at least 2 weeks.

The method or kit of any of the foregoing clauses wherein the agent is cefazolin or a salt thereof, and the administering thereof is discontinued for a predetermined period of time.

The method or kit of any of the foregoing clauses wherein the agent is cefazolin or a salt thereof administered for 1 week.

The method or kit of any of the foregoing clauses wherein the agent is cefazolin or a salt thereof administered for 2 weeks.

The method or kit of any of the foregoing clauses wherein the agent is ceftriaxone or a salt thereof intravenously administered at a dose of 1-2 g q24 h.

The method or kit of any of the foregoing clauses wherein the agent is cefazolin or a salt thereof administered for at least 1 week.

The method or kit of any of the foregoing clauses wherein the agent is cefazolin or a salt thereof administered for at least 2 weeks.

The method or kit of any of the foregoing clauses wherein the agent is cefazolin or a salt thereof, and the administering thereof is discontinued for a predetermined period of time.

The method or kit of any of the foregoing clauses wherein the agent is cefazolin or a salt thereof administered for 1 week.

The method or kit of any of the foregoing clauses wherein the agent is cefazolin or a salt thereof administered for 2 weeks.

Additional illustrative embodiments include methods and kits described herein wherein the agent is parenterally administered and:

wherein the agent is vancomycin and an illustrative dose of vancomycin for a human host animal is from about 12 mg/kg to about 18 mg/kg, from about 14 mg/kg to about 16 mg/kg, or about 15 mg/kg administered every 12 hours;

wherein the agent is daptomycin and an illustrative dose for a human host animal of daptomycin is from about 3 mg/kg to about 9 mg/kg, from about 5 mg/kg to about 7 mg/kg, or about 6 mg/kg administered every 24 hours for about 7 to about 14 days;

wherein the agent is nafcillin and an illustrative dose for a human host animal of nafcillin is from about 15 mg/kg to about 40 mg/kg, from about 18 mg/kg to about 18 mg/kg, or from about 1.5 g to about 2 g administered every 4 to 6 hours;

wherein the agent is cefazolin and an illustrative dose for a human host animal of cefazolin is from about 10 mg/kg to about 36 mg/kg, from about 12 mg/kg to about 30 mg/kg, or from about 1 g to about 2 g administered every 8 hours;

wherein the agent is ceftriaxone and an illustrative dose for a human host animal of ceftriaxone is from about 10 mg/kg to about 36 mg/kg, from about 12 mg/kg to about 30 mg/kg, or from about 1 g to about 2 g administered every 24 hours;

wherein the agent is dalbavancin and an illustrative dose for a human host animal of dalbavancin is from about 600 mg to about 1300 mg, from about 700 mg to about 1200 mg/kg or about 1000 mg administered once in week, followed by from about 400 mg to about 600 mg or about 500 mg administered once in second week;

wherein the agent is oritavancin and an illustrative dose for a human host animal of is oritavancin from about 1000 mg to about 1400 mg, from about 1100 mg to about 1300 mg, or about 1200 mg administered once;

wherein the agent is telavancin and an illustrative dose for a human host animal of telavancin is from about 6 mg/kg to about 14 mg/kg, from about 8 mg/kg to about 12 mg/kg, or about 10 mg/kg administered every 24 hours for about 7 to about 14 days; and/or wherein the agent is linezolid and an illustrative dose for a human host animal of linezolid is from about 4 mg/kg to about 12 mg/kg, from about 5 mg/kg to about 10 mg/kg or about 600 mg administered every 12 hours for from about 10 to about 14 days. Linezolid can be administered by injection, IV or administered orally.

Additional illustrative embodiments include methods and kits described herein wherein the agent is orally administered and:

wherein the agent is cephalexin and an illustrative dose for a human host animal of cephalexin is from about 5 mg/kg to about 15 mg/kg, from about 6 mg/kg to about 10 mg/kg, or about 500 mg administered 3 times/day or 4 times/day;

wherein the agent is dicloxacillin and an illustrative dose for a human host animal of dicloxacillin is from about 5 mg/kg to about 15 mg/kg, from about 6 mg/kg to about 10 mg/kg, or about 500 mg administered 3 times/day or 4 times/day;

wherein the agent is minocycline and an illustrative dose for a human host animal of minocycline is from about 1 mg/kg to about 2 mg/kg, from about 1.2 mg/kg to about 1.6 mg/kg, or about 100 mg administered 2 times/day;

wherein the agent is co-trimoxazole (trimethoprim 160 mg+sulfamethoxazole 800 mg) and an illustrative dose for a human host animal of co-trimoxazole is from about 2.0 mg/kg to about 2.7 mg/kg (based on the amount of trimethoprim in the mixture), from about 2.1 mg/kg to about 2.5 mg/kg (based on the amount of trimethoprim in the mixture), or about trimethoprim 160 mg+sulfamethoxazole 800 mg administered every 8 hours; and/or wherein the agent is tedizolid and an illustrative dose for a human host animal of tedizolid is from about 3 mg/kg to about 4 mg/kg, from about 2.5 mg/kg to about 3.5 mg/kg, or about 200 mg administered once daily for about 6 days.

As used herein, salts of pharmaceutical ingredients described herein are illustratively pharmaceutically acceptable salts.

As used herein, twice daily refers to either, or both, b.i.d. and/or q12 h.

In another illustrative embodiment, methods and kits are described herein for treating BJIs caused by both methicillin-sensitive S. aureus (MSSA) and methicillin-resistant Staphylococcus aureus (MRSA), and/or coagulase-negative staphylococcal (CoNS), including but not limited to, S. epidermidis, S. hominis, or S. haemolyticus.

In another embodiment, methods an kits are described herein for treating BJIs that include co-infection with other organisms, such as but not limited to, Corynebacterium spp., Propionibacterium acnes, beta-hemolytic Streptococci, and Enterococci, and the like. In another embodiment, the first dose of FA on the first day is about 1000-1500 mg BID, about 1100-1500 mg BID, or about 1200-1500 mg BID. In another embodiment, the first dose of FA on the first day is about 1100 mg BID. In another embodiment, the second and each subsequent dose of FA is 600 mg BID. In another embodiment, the method comprises administering FA for a total of about 60 days, about 90 days, about 120 days, about 180 days, and/or about 365 days.

In another embodiment, the method comprises the steps of parental administration of oritavancin or a pharmaceutically acceptable salt thereof on a first day, and daily oral administration of FA on the first though at least the fifteenth day. In one variation, the method comprises the steps of parental administration of oritavancin or a pharmaceutically acceptable salt thereof on a first day, and daily oral administration of FA on the second though at least the fifteenth day.

In another embodiment, the method comprises the steps of parental administration of oritavancin or a pharmaceutically acceptable salt thereof on a first day, daily oral administration of FA on the first though fourteenth day, parental administration of oritavancin or a pharmaceutically acceptable salt thereof on the fifteenth day, and daily oral administration of FA on the fifteenth though at least the thirtieth day. In one variation, the method comprises the steps of parental administration of oritavancin or a pharmaceutically acceptable salt thereof on a first day, daily oral administration of FA on the second though fourteenth day, parental administration of oritavancin or a pharmaceutically acceptable salt thereof on the fifteenth day, and daily oral administration of FA on the fifteenth though at least the thirtieth day.

In another embodiment, the method does not include the co-administration of rifampicin. It is generally accepted that the co-administration of rifampicin is necessary to guard against resistance development arising from administration of fusidic acid. It has been surprisingly discovered herein that resistance management in conjunction with FA administration may not be necessary, and in any case, is equally well managed by the co-administration of the one or more agents described herein. For example, without being bound by theory, it is believed herein that because oritavancin is bactericidal, the potential for resistance development using a follow-on administration of FA is low. Without being bound by theory, it is also believed herein that because oritavancin has a long half-life, such as a half-life of greater than 10 days, or about 15 days, the potential for resistance development using a follow-on administration of FA is also low. It has also been surprisingly discovered herein that resistance management in conjunction with FA administration may not be necessary, and in any case, is equally well managed by the co-administration of an oxazolidinone antibiotic, such as linezolid or a pharmaceutically acceptable salt thereof.

It has been reported that one of the challenges to treating bone and joint infections, and in particular, bone and joint infections in conjunction with prostheses is the development biofilms. Such biofilms are reported to decrease the efficacy of antibiotic treatment by a variety of mechanisms.

It has been discovered herein that FA improves the penetration of oritavancin and pharmaceutically acceptable salts thereof across bacterial biofilms. It has also been discovered herein that oritavancin improves the penetration of FA and pharmaceutically acceptable salts thereof across bacterial biofilms.

In another embodiment, the infection is an ABSSSI. In another embodiment, the infection is caused at least in part by a gram positive bacteria.

In another embodiment, the bacterial infection is an infection caused at least in part by bacteria selected from the group consisting of staphylococci, including coagulase-negative staphylococci and coagulase-positive staphylococci, streptococci, including Group A beta hemolytic streptococci, non-Group A beta hemolytic streptococci and viridans group streptococci, enterococci, Nesseria species, Clostridium species, Bordetella species, Bacillus species and Corynebacterium species. Preferably, the bacterial infection is an infection caused by bacteria selected from the group consisting of Staphylococcus aureus (methicillin-resistant and -susceptible), Staphylococus epidermidis, Staphylococus hemolyticus, Staphylococus saprophyticus, Staphylococus lugdunensis, Staphylococus capitis, Staphylococus caprae, Staphylococus saccharolyticus, Staphylococus simulans, Staphylococus warneri, Staphylococus hominis, Staphylococus intermedius, Staphylococcus pseudointermedius, Staphylococus lyricus, Streptococcus pyogenes, Streptococcus agalactiae, Streptococcus dysgalactiae subspecies dysgalactiae, Streptococcus anginosus, Streptococcus mitis, Streptococcus salivarius, Streptococcus bovis, Streptococcus mutans, Neisseria gonorrhoeae, Neisseria meningitidis, Bacillus anthracis, Bordetella pertussis, Clostridium difficile, Enterococcus faecalis, Enterococcus faecium and Corynebacterium diphtheriae. In particular aspects, the bacterial infection is an infection caused by Enterococcus faecalis or Enterococcus faecium.

In another embodiment, the infection is caused at least in part by Staphylococcus spp.

In another embodiment, the infection is caused at least in part by S. aureus.

In another embodiment, the infection is caused at least in part by MRSA.

Additional illustrative doses and doing protocols are described in U.S. Pat. No. 8,450,300, the entire disclosure of which is incorporated herein by reference. It is to be understood that as used herein in describing the various compositions, kits, and methods, the term “fusidic acid” or “FA”, unless otherwise indicated, generally refers to the hemihydrate form of the compound, as well as pharmaceutically acceptable salts, other hydrates, solvates, or mixtures thereof. It is to be further understood that such compositions, kits, and methods may include fusidic acid alone, the hemihydrate form of the compound alone, any such pharmaceutically acceptable salt, other hydrate, or solvate thereof alone, or any mixtures of the foregoing, including sodium fusidate.

As used herein, the term “pharmaceutically acceptable salt” generally refers to non-toxic base addition salts derived from inorganic and organic bases. Illustrative base addition salts include, but are not limited to, those derived from inorganic bases, such as ammonium or alkali or alkaline earth metal hydroxides, carbonates, bicarbonates, and the like, as well as alkylamine and organic amino salts, such as an ethanolamine salt. Further illustrative base addition salts include, but are not limited to, sodium hydroxide, potassium hydroxide, ammonium hydroxide, potassium carbonate, sodium carbonate, sodium bicarbonate, potassium bicarbonate, calcium hydroxide, calcium carbonate, and the like. In another embodiment, the base addition salts are potassium and sodium salts. In another embodiment, the base addition salt is a sodium salt. In another embodiment, sodium fusidate, also termed CEM-102 herein, having the following structure

It is to be understood that the particular counter-ion forming a part of any salt described herein is not of a critical nature, so long as the salt as a whole is pharmacologically acceptable and as long as the counter-ion does not contribute undesired qualities to the salt as a whole.

As used herein, the term “solvates” refers to compounds described herein complexed with a solvent molecule. It is appreciated that compounds described herein may form such complexes with solvents by simply mixing the compounds with a solvent, or dissolving the compounds in a solvent. It is appreciated that where the compounds are to be used as pharmaceuticals, such solvents are pharmaceutically acceptable solvents. It is further appreciated that where the compounds are to be used as pharmaceuticals, the relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In some embodiments, the solvates are amorphous, and in other embodiments, the solvates are crystalline.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

In addition, in those embodiments described herein drawn to combination therapy comprising administration of a second and/or third agent, and FA or a pharmaceutically acceptable salt thereof, “therapeutically effective amount” refers to that amount of the combination of agents taken together so that the combined effect elicits the desired biological or medicinal response. For example, the therapeutically effective amount of the second and/or third agent, and FA or a pharmaceutically acceptable salt thereof, would be the amount of second and/or third agent, and the amount of FA or a pharmaceutically acceptable salt thereof that when taken together or sequentially have a combined effect that is therapeutically effective. Further, it is appreciated that in some embodiments of such methods that include co-administration, that co-administration amount of a second and/or third agent, or FA or a pharmaceutically acceptable salt thereof when taken individually may or may not be therapeutically effective.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes local use, such as when administered locally to the site of disease, injury, or defect, or to a particular organ or tissue system. Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the host animal being treated. It is further appreciated that local administration may be directly in the injury site, or locally in the surrounding tissue.

Depending upon the disease as described herein, the route of administration and/or whether the compounds and/or compositions are administered locally or systemically, a wide range of permissible dosages are contemplated herein. The dosages may be single or divided, and may administered according to a wide variety of protocols, including q.d., b.i.d., t.i.d., or even every other day, once a week, twice a month, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

The following examples further illustrate specific embodiments of the invention; however, the following illustrative examples should not be interpreted in any way to limit the invention. The figures and drawings described hereinbelow are set forth in U.S. Pat. No. 8,450,300, and specifically incorporated herein by reference.

### EXAMPLES

### Example 1—Study of the Spectrum of CEM-102 (Fusidic Acid) Against Contemporary Wildtype (WT) Bacterial Species Including Mutational Resistance (R) Analysis, and Synergy Testing

A collection of 114 WT isolates (>80 species) was used to define the contemporary limits of CEM-102 (fusidic acid; FA) spectrum against Gram-positive (GP) and −negative (GN) species. CLSI broth microdilution (BMD) and anaerobic agar dilution (AD) methods were performed. Modifications of standard test methods included adding 10% human serum, adjusting the medium pH to 5, 6, and 8, and synergy was assessed by the checkerboard method. Mutational rates to R were determined at 4×, 8× and 16×MIC.

Against GP, FA MIC values ranged from 0.06-32 μg/ml with greatest potency against S. aureus, Corynebacterium spp. and M. luteus (MIC results 0.25, ≦0.12 and ≦0.5 μg/ml, respectively). Enterococci and streptococci were less susceptible (S; MIC ranges of 2-8 and 16-32 μg/ml, respectively).

When tested against 217 S. aureus Canadian isolates, the MIC₅₀ for CEM-102 was 0.12 μg/mL and the MIC₉₀ was 0.25 μg/mL, which is only slightly higher than strains in the US (MIC₉₀ 0.12 μg/mL). The MIC population distribution study showed that only 6.5% of the Canadian S. aureus isolates had MICs of 2.0 μg/mL and would be classified as resistant. By comparison, ciprofloxacin, clindamycin, and erythromycin resistance rates were 41.5, 30.9, and 52.1% respectively). This is based upon established breakpoints of greater than or equal to 2 ug/ml (more strains are expected to be classified as susceptible in view of the dosing regimes disclosed in the present application due to the much higher blood levels of FA; many resistant strains, with MICs of 4-32 ug/ml, are expected to be susceptible under the new dosing regimens).

FA activity against GN species was limited (all MIC values ≧2 μg/ml) except for E. brevis, M. catarrhalis and N. meningitidis (MICs, 0.12-0.5 μg/ml). A 4-fold increase in FA MIC results was observed when 10% serum was added. Decreasing medium pH to 5.0-6.0 negated the protein binding effects. Among the 8 combinations of drugs tested, no antagonism was observed with FA (Table 1). Single-step mutational rates ranged from 1.2×10⁻⁶ for 4×MIC to 9.8×10⁻⁸ for 16×MIC.

TABLE 1

| | Synergy | | Addi- | Indif- | Antag- | Indeter- |
|---|---|---|---|---|---|---|
| FA/co-drug | Complete | Partial | tive | ferent | onism | minate |
| Rifampin | 0 | 5 | 1 | 0 | 0 | 0 |
| Levofloxacin | 0 | 0 | 0 | 4 | 0 | 2 |
| Gentamicin | 1 | 1 | 3 | 1 | 0 | 0 |
| Oxacillin | 0 | 1 | 1 | 3 | 0 | 1 |
| Vancomycin | 0 | 0 | 2 | 4 | 0 | 0 |
| All agents | 1 | 7 | 9 | 24 | 0 | 7 |
| tested | | | | | | |

FA demonstrated potent GP activity, especially against the staphylococci. A more limited activity was observed against GN species isolates. Added serum proteins adversely influenced MIC values; however lower media pH like seen at infection sites decreased negative protein binding effects. FA in vitro activity was most improved when combined with RIF.

### Example 2—Antimicrobial Activity of CEM-102 (Fusidic Acid) Against Canadian Isolates of Staphylococci and Streptococci

To determine a contemporary susceptibility (S) spectrum pattern, 153 GP isolates (123 S. aureus, 15 coagulase-negative staphylococci [CoNS] and 15 S. pyogenes [SPYO]) were collected from 5 Canadian medical centers between 2001 and 2006. Reference broth microdilution (BMD) S testing was performed by CLSI M07-A8, 2009 methods for FA and 13 comparator antimicrobials. FA MIC results for S. aureus had MIC₅₀ and MIC₉₀ values of 0.12 μg/mL for the 2001-2002 and 2003-2004 time periods, however, for 2005-2006 the MIC₉₀ increased to ≧2 μg/ml (Table 2). Applying an international breakpoint from literature reviews at ≦0.5 μg/ml (S) and ≧2 μg/ml (R), the S. aureus isolates showed a small increase in the R rate over time (5.0-12.2%), now confirmed by 2007-2008 results. The overall S. aureus population had a MIC₉₀ of 0.25 μg/ml and R rate of 8.1%. Some comparator agents showed higher R rates that remained stable over the period tested with highest R noted for erythromycin (ERY, 52.0%), ciprofloxacin (43.9%), and clindamycin (CLI, 28.5%). CoNS isolates had FA MIC₅₀ and MIC₉₀ values at 0.12 and 16 μg/ml, respectively. SPYO isolates were only moderately S to FA with all values at 4 or 8 μg/ml. Among the comparator agents, ERY had an R rate of 20.0% and CLI at 13.3% for SPYO.

TABLE 2

| S. aureus | No. inhibited at MIC (μg/mL) of: | | | | | | | |
|---|---|---|---|---|---|---|---|---|
| (years tested) | ≦0.03 | 0.06 | 0.12 | 0.25 | 0.5 | 1 | ≧2 | % at ≦0.5^{a} |
| 2001-2002 | — | 8 | 29 | 1 | — | — | 2 | 95.0 |
| 2003-2004 | — | 6 | 33 | — | — | — | 3 | 92.9 |
| 2005-2006 | — | 2 | 32 | 2 | — | — | 5 | 87.8 |

^{a}6.0% R for 2007-2008

FA demonstrated potent activity against Canadian staphylococci isolates with a low overall R rate (8.1%) among S. aureus. CoNS isolates had a greater R rate than S. aureus. FA had a narrow range of MIC results (4-8 μg/ml) and was less active against SPYO.

### Example 3—In Vitro Activity of CEM-102 (Fusidic acid) Against Resistant Strains of Staphylococcus aureus

The activity of CEM-102 (fusidic acid) against a variety of resistant strains of Staphylococcus aureus was investigated. The in vitro activity of CEM-102 was compared with that of telithromycin, azithromycin, erythromycin, levofloxacin, linezolid, and doxycycline against a total of 145 resistant S. aureus by agar dilution procedures (CLSI, M7-A7, M100-S18). The tested strains included S. aureus MRSA (Mec A genotype; 100 isolates), macrolide-resistant (ermA, B, C genotype or MLSb-resistant; 25), and ciprofloxacin-resistant (gyrA and parC genotype; 20).

Against S. aureus MRSA (MecA), CEM-102 (MIC₉₀, 0.25 mg/L) and telithromycin (MIC₉₀ 0.06 mg/L) were more active than doxycycline (MIC₉₀ 1 mg/L), linezolid (MIC₉₀ 2 mg/L), levofloxacin (MIC₉₀ 16 mg/L), azithromycin (MIC₉₀>32 mg/L) and erythromycin (MIC₉₀>32 mg/L). CEM-102 (MIC₉₀ 0.25 mg/L) was significantly superior to linezolid (MIC₉₀ 2 mg/L), levofloxacin (MIC₉₀ 4 mg/L), telithromycin (MIC₉₀ 4 mg/L), azithromycin (MIC₉₀>32 mg/L), and erythromycin (MIC₉₀>32 mg/L) against macrolide-resistant S. aureus (ermA, B, C genotype or MLSb-resistant). Against ciprofloxacin-resistant (gyrA and parC genotype) S. aureus, erythromycin (MIC₉₀>32 mg/L), levofloxacin (MIC₉₀>32 mg/L), azithromycin (MIC₉₀, 16 mg/L), linezolid (MIC₉₀, 2 mg/L), and doxycycline (MIC₉₀ 1 mg/L) were less active than CEM-102 (MIC₉₀ 0.25 mg/L) and telithromycin (MIC₉₀, 0.06 mg/L).

These data confirm the activity of CEM-102 against resistant S. aureus and that cross resistance with other classes of antibiotics is not observed.

### Example 4—Single Oral Dose Bioavailability Study of CEM-102 (Sodium Fusidate)

The primary objective of the study was to evaluate the relative bioavailability of single oral doses of CEM-102 (sodium fusidate) 500 mg (2×250 mg) tablets and Fucidin® (sodium fusidate) 500 mg (2×250 mg) tablets in healthy subjects in a fed or fasted state. This was a single-center, Phase 1, double-blind, randomized, 3 period, fed/fasted crossover bioequivalence study designed to evaluate the relative bioavailability and safety and tolerability of a single oral dose of CEM-102 500 mg compared to Fucidin® 500 mg in healthy subjects. Subjects were randomized in equal numbers to the 4 treatment sequences (Table 3).

TABLE 3
Treatment Sequences

| Treatment | Subjects | | | |
|---|---|---|---|---|
| Sequence | Randomized | Study Period 1 | Study Period 2 | Study Period 3 |
| 1 | 7 | Fucidin ® 500 mg | CEM-102 500 mg | Fucidin ® 500 mg |
| | | (2 × 250 mg) | (2 × 250 mg) | (2 × 250 mg) |
| | | Fasting | Fasting | Fed |
| 2 | 7 | Fucidin ® 500 mg | CEM-102 500 mg | CEM-102 500 mg |
| | | (2 × 250 mg) | (2 × 250 mg) | (2 × 250 mg) |
| | | Fasting | Fasting | Fed |
| 3 | 7 | CEM-102 500 mg | Fucidin ® 500 mg | Fucidin ® 500 mg |
| | | (2 × 250 mg) | (2 × 250 mg) | (2 × 250 mg) |
| | | Fasting | Fasting | Fed |
| 4 | 7 | CEM-102 500 mg | Fucidin ® 500 mg | CEM-102 500 mg |
| | | (2 × 250 mg) | (2 × 250 mg) | (2 × 250 mg) |
| | | Fasting | Fasting | Fed |

Study Period 1, 2, and 3 (Days 1, 15, and 29): One dose of study drug 500 mg (2×250 mg) of CEM-102 or Fucidin® was given at 8:00 AM (˜30 minutes). On Day 1 of Study Periods 1 and 2 (Days 1 and 15), subjects fasted overnight for at least 10 hours prior to dosing. On Day 1 of Study Period 3 (Day 29), subjects were given a high fat caloric meal which was to be entirely consumed within 30 minutes prior to study drug administration.

Blood samples for assay of plasma concentrations were collected on Days 1, 15 and 29 at pre-dose and 0.5, 1, 1.5, 2, 2.5, 3, 3.5, 4, 6, 8, 12, 16, 24, 36, and 48 hours after the dose. Plasma concentrations were assayed at MicroConstants, Inc. in San Diego, Calif. using a validated liquid chromatography with dual tandem mass spectrometry (LC/MS/MS) method with a lower limit of quantitation (LLOQ) of 20.0 ng/mL

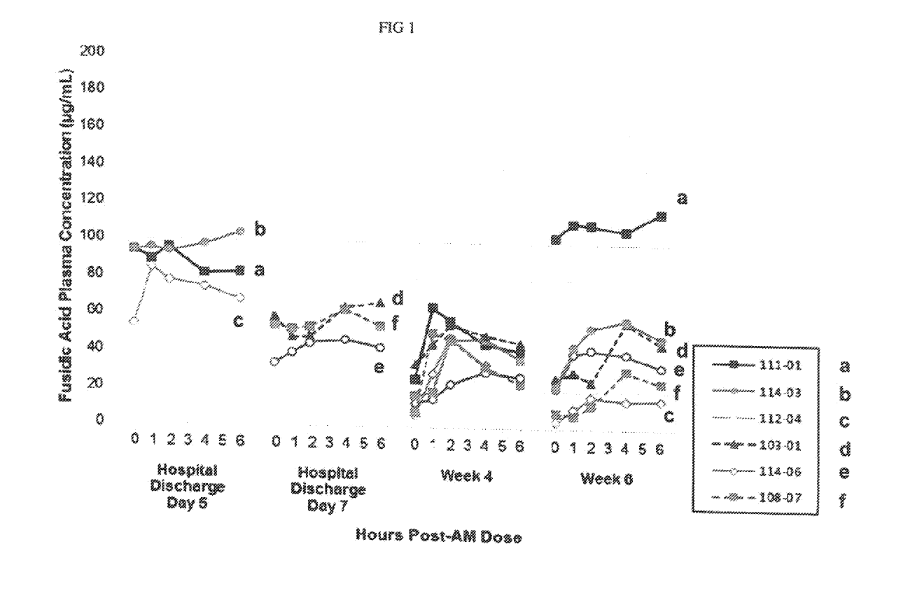

Mean plasma concentrations of fusidic acid are depicted in FIGS. 1 and 2 on semi-log and linear scales. The median (minimum-maximum) tₘₐₓ values for fusidic acid for the CEM-102 and Fucidin® products under fasting conditions were 3.00 hours (1.00 to 4.00 hours) and 2.52 hours (1.00 to 6.00 hours), respectively.

Descriptive statistics of pharmacokinetic results for fusidic acid in plasma are presented in Table 4.

TABLE 4
Descriptive Statistics for the Assessment of Food-effect and Fed Pharmacokinetics

| | | CEM-102 | | CEM-102 | | Fucidin ® | |
|---|---|---|---|---|---|---|---|
| | | Fed | | Fasting | | Fed | |
| | Parameter | (C) | | (A) | | (D) | |
| Geometric Mean (CV %) | AUC₀₋ₜ (ng · h/mL) | 268561 | (31.5) | 300352 | (30.9) | 264713 | (40.5) |
| | AUCᵢₙₜ (ng · h/mL) | 285056 | (32.7) | 318391 | (32.0) | 280680 | (40.5) |
| | Cₘₐₓ (ng/mL) | 21175 | (25.2) | 27413 | (23.8) | 21541 | (34.0) |
| Arithmetic Mean ± SD | t_{1/2} (h) | 11.5 ± 2.75 | | 11.8 ± 2.20 | | 11.5 ± 2.62 | |
| Median (Min-Max) | tₘₐₓ (h) | 3.50 | (1.50-6.00) | 3.00 | (1.00-4.00) | 4.00 | (1.00-8.25) |

Food appeared to decrease the Cmax of CEM-102 by approximately 23%. However, the total exposure (AUC), the time to peak plasma concentrations and the half-life were comparable when the CEM-102 500 mg dose was administered under fed and fasting conditions (approximately 285 vs. 318 μg·h/mL, 3.50 vs. 3.00 hours and 11.5 vs. 11.8 hours, respectively)

### Example 5—Multi-Dose Bioavailability Study Using FA at 500 mg TID

The pharmacokinetics of multiple oral doses of CEM-102 (sodium fusidate) 500 mg tablets in healthy subjects was evaluated. Subjects were randomized in a 3:1 ratio and received either CEM-102 500 mg (2×250 mg; n=18) or placebo tablets TID (n=6) for 4.5 days for a total of 13 doses. Blood samples for assay of plasma concentrations of CEM-102 and placebo were collected pre-dose on Day 1 and at 24, 48, 72, and 96 hours. Samples were also collected at the following times after the morning dose on Day 5: 0.5, 1, 1.5, 2, 2.5, 3, 3.5, 4, 6, 8, 12, 16, 24, 36, and 48 hours. Plasma concentrations of CEM-102 were assayed at MicroConstants, Inc. in San Diego, Calif., USA using a validated LC/MS/MS method with a lower limit of quantitation (LLOQ) of 20.0 ng/mL. Mean plasma concentrations of CEM-102 on Day 5 (13 doses) are depicted on semi-log (FIG. 3) and linear scales (FIG. 4). Descriptive statistics of pharmacokinetic results for CEM-102 in plasma are presented in Table 5.

TABLE 5

| | | CEM-102 in Plasma | |
|---|---|---|---|
| Parameter | | (Day 5) | |
| Geometric Mean | AUC0-tss (ng · h/mL) | 3562344 | (34.7) |
| (CV %) | AUC0-τss (ng · h/mL) | 1030827 | (26.3) |
| | Cmaxss (ng/mL) | 145680 | (25.3) |
| | Cmin (ng/mL) | 106600 | (25.5) |
| Arithmetic Mean ± SD | t½ ss (h) | 18.7 ± 5.30 | |
| Median (Min-Max) | tmaxss (h) | 3.00 | (0.50-6.00) |

On Day 5, the PK profile of CEM-102 in plasma following repeated oral doses of 500 mg TID was well characterized. Mean plasma concentrations of CEM-102 remained above the LLOQ (20.0 ng/mL) throughout the sampling schedule. Mean CEM-102 concentrations rose rapidly and peaked at approximately 3 hours (range 0.5-6 hours) post-dose and then declined slowly with an apparent half-life of approximately 19 hours. Maximum plasma concentrations ranged between 87.6 and 245 ug/mL. Trough levels rose steadily from Days 1 through 5 attaining 105 ug/mL prior to dosing on Day 5 and 120 ug/mL at 8 hours post-dose. This continuing rise in mean trough concentrations following the last dose may indicate that steady-state was not yet reached by Day 5.

The pharmacokinetic profile of CEM-102 in plasma following multiple oral doses of 500 mg administered TID for 13 doses over 4.5 consecutive days was well characterized. However, the continuing rise in mean trough concentrations following the last dose may indicate that steady-state was not yet reached by Day 5. The results of this study supported the published reports that a steady state high level of FA is not reached until after the 4th day using the standard dosing regimen of 500 mg TID.

### Example 6—Dose Escalation Study Using Single- and Multi-Dose FA

The pharmacokinetics (PK), safety, and tolerability of single and multiple doses of CEM-102 (sodium fusidate) were evaluated in a single-center, Phase 1, double-blind, randomized, placebo-controlled, dose-escalating study in 32 healthy adult subjects enrolled in 4 dosage groups (550, 1100, 1650, and 2200 mg). In each cohort, six subjects were to receive CEM-102 and two were to receive placebo in a single dose (Period 1) and then multiple doses BID for a total of 11 doses over 5.5 days (Period 2). The two dosing periods were separated by a seven-day washout period between the single dose in Period 1 and the first dose in Period 2. Dosing proceeded as planned for Cohorts 1 to 3; however, Cohort 4 received only the single dose of 2200 mg (Period 1), because dose-limiting gastrointestinal intolerance was observed after multiple doses of 1650 mg BID in Cohort 3. Period 2 for Cohort 4 evaluated an initial loading dose regimen, and a second loading dose regimen was evaluated in an additional cohort (Cohort 5) per a protocol amendment.

CEM-102 was considered safe and generally well tolerated. No serious or life-threatening adverse events (AEs) occurred. Seventy-four AEs were reported in 19 of the 32 study subjects, all of which were mild or moderate in severity. Of the 70 treatment-emergent AEs reported all except 7 were in Period 2, in which subjects received multiple daily doses over 5.5 days. Most of the AEs were considered possibly related to study drug.

Single doses of CEM-102 up to 2200 mg were well tolerated. The only AEs reported in the 550 mg and 1100 mg cohorts were in subjects on placebo and no AEs were reported in the 1650 mg cohort. As described in product information documents from countries in which sodium fusidate is approved and from published reports, gastrointestinal AEs are the most commonly reported AEs associated with administration of oral sodium fusidate. In the present study, no gastrointestinal AEs were reported in the 550, 1100, or 1650 mg cohorts (Table 6). Nausea and vomiting occurred in 1 subject after the single dose of 2200 mg and nausea alone occurred in 2 others; therefore, gastrointestinal intolerance appeared to be dose-related with a threshold between 1650 and 2200 mg. There were no clinically significant changes in physical examinations, vital signs, electrocardiograms (ECGs), or laboratory parameters after single doses of CEM-102.

As with single doses, the most common AE after multiple doses of CEM-102 was nausea, reported in 9 subjects (550 mg, 2 subjects; 1100 mg, 2 subjects; 1650 mg, 5 subjects) (Table 7). Nausea occurred most frequently after 3 to 5 days of dosing with 1650 mg BID. No pharmacologic treatment was required for most of the AEs.

Four of the subjects at the 1650 mg multiple dose level also experienced vomiting (Grade 1 in 3 subjects and Grade 2 in 1 subject). Although the vomiting was considered mild in 3 of the 4 subjects, the decision was made to limit evaluation of multiple doses to the 1650 mg dose level. Consequently, escalation to the 2200 mg dose in Cohort 4 involved only Period 1 with a single dose of study drug. PK results from single and multiple dose administration of CEM-102 are shown in Table 8 and FIG. 5.

TABLE 8
CEM-102 Single and Multiple Dose Pharmacokinetic Parameters

| | Single and Multiple Dose Groups | | | | | | Single Dose Only | |
|---|---|---|---|---|---|---|---|---|
| | Cohort 1 | | Cohort 2 | | Cohort 3 | | Cohort 4 | |
| | 550 mg | | 1100 mg | | 1650 mg | | 2200 mg | |
| Parameter | Mean | SD | Mean | SD | Mean | SD | Mean | SD |

Period 1 Day 1

| | | | | | | | | |
|---|---|---|---|---|---|---|---|---|
| Cₘₐₓ, μg/mL | 33.4 | 12.2 | 72.2 | 10.8 | 102 | 25.8 | 128 | 28.3 |
| Tₘₐₓ, h^{a} | 2.00 | (2-3) | 3.50 | (1-4) | 3.00 | (2-4) | 6.00 | (3-8) |
| Kₑₗ, h^{-1b} | 0.0564 | 0.0130 | 0.0617 | 0.0120 | 0.0498 | 0.0151 | 0.0500 | 0.0159 |
| T_{1/2}, h^{c} | 12.3 | 2.98 | 11.2 | 2.17 | 13.9 | 4.29 | 13.9 | 4.52 |
| AUC₍₀₋₂₄₎, μg · h/mL | 242 | 102 | 844 | 115 | 1,260 | 386 | 1,690 | 427 |
| AUC_{(0-inf)}, μg · h/mL | 441 | 269 | 1,100 | 247 | 1,800 | 689 | 2,650 | 978 |
| CL/F, L/h | 1.69 | 1.00 | 1.04 | 0.202 | 1.07 | 0.519 | 0.924 | 0.434 |
| V_{d}/F, L | 28.4 | 10.8 | 16.9 | 1.20 | 21.1 | 4.96 | 19.2 | 6.38 |

Period 2 Day 6

| | | | | | | | | |
|---|---|---|---|---|---|---|---|---|
| Cₘₐₓ, μg/mL | 130 | 30.5 | 281 | 52.5 | 324 | 26.8 | — | — |
| Tₘₐₓ, h^{a} | 3.00 | (1.5-4) | 4.00 | (4-8) | 4.00 | (1.5-6) | — | — |
| Kₑₗ, h^{-1b} | 0.0554 | 0.0131 | 0.0404 | 0.0162 | 0.0199 | 0.0118 | — | — |
| T_{1/2}, h^{c} | 12.5 | 3.05 | 17.1 | 6.79 | 31.6 | 18.9 | — | — |
| AUC₍₀₋₁₂₎, μg · h/mL | 1,150 | 433 | 2,530 | 417 | 3290 | 146 | — | — |
| CL/F, L/h | 0.553 | 0.255 | 0.449 | 0.100 | 0.0503 | 0.0223 | — | — |
| V_{d}/F, L | 9.88 | 3.05 | 12.7 | 5.66 | 34.8 | 22.9 | — | — |
| Cₘₐₓ accumulation ratio ^{d} | 3.89 | — | 3.89 | — | 3.18 | — | — | — |
| AUC₍₀₋₁₂₎ accumulation | 2.61 | — | 2.30 | — | 1.83 | — | — | — |
| ratio ^{e} | | | | | | | | |

^{a}Expressed as median and range
^{b}Apparent first-order terminal elimination rate constant
^{c}Expressed as harmonic mean and pseudo SD
^{d} Mean Cₘₐₓ Day 6/Mean Cₘₐₓ Day 1
^{e} Mean AUC₍₀₋₁₂₎ Day 6/Mean AUC_{(0-inf)} Day 1

### Example 7—Pharmacodynamics of CEM-102 Against Methicillin-Resistant Staphylococcus aureus Using In Vitro Models

A hollow fiber model was used to evaluate CEM-102 resistance potential in methicillin-resistant S. aureus (MRSA) strain USA 300 (Network of Antimicrobial Resistance in Staphylococcus aureus (NARSA), Chantilly, Va.). USA 300 is a highly virulent strain of MRSA and is the most common community-associated MRSA isolate in the USA. MIC values were determined in accordance with Clinical and Laboratory Standards Institute (CLSI).

The hollow fiber model comprised a two-compartment hollow fiber model (Louie et al. Antimicrob Agents Chemother 52:2486-2496(2008)) consisting of a volume of 15 mL in the central compartment, with multiple ports for the removal of broth, delivery of antibiotics, and collection of bacterial and antimicrobial samples. A peristaltic pump was used to continually replace antibiotic-containing medium with fresh media (Mueller Hinton Broth, supplemented with calcium, magnesium, and human albumin to a final concentration of 4 g/dL, simulating human physiologic levels) at a rate to simulate the half-life of CEM-102 based on human PK data. All experiments were performed in duplicate.

Hollow fibers contain 15 ml of media was prepared and inoculated with 10⁶ colony forming units (CFU)/mL USA 300 bacteria. One of three different dosing regimens of CEM-120 was then applied to the fibers: (1) 600 mg/ml q12 h; (2) 1200 mg/ml×2, followed by 600 mg/ml, q12 h; (3) 1500 mg/ml×2, followed by 600 mg/ml, q12 h.

On Days 0, 1, 2, 3, 4, 5, 6, 7, 8, 9 and 10 samples were withdrawn from the fibers and plated on media with no-drug (TSA II with 5% sheep blood) or drug-containing agar (brain heart infusion), consisting of 4, 8, 16×MIC of CEM-102 (1 ug/ml, 2 ug/ml, 4.0 ug/ml, respectively; MIC=0.25 ug/ml). Treatment using 600 mg FA q12 h resulted in regrowth and development of resistance by 24 h and 48 h. Treatment using 1200 mg×2 then 600 mg q12 h suppressed bacterial counts and reduced bacterial counts by ˜2 logs, with rebound at 72 h. Treatment using 1500 mg×2 then 600 mg q12 h suppressed resistance until >72 h and resulted in killing over 2.5 log and even approached the threshold of bactericidal activity, and rebounded at 144 h.

### Example 8—In Vivo Evaluation of Co-Administration of FA and RIF I Human Host Animals

Fusidic acid (CEM-102) was administered orally with a loading dose of 1500 mg twice daily (BID) or 3000 mg total daily dose (TDD) on Study Day 1, followed by 1500 mg TDD (900 mg in the morning and 600 mg in the evening, or vice versa) thereafter. Temporary CEM-102 dose reduction to a 1200 mg TDD (or 600 mg BID) in response to poor tolerability (symptoms or safety laboratory abnormalities) of therapy was permitted before or after randomization.

Based on dosing results observed in a prior Phase 1 pharmacokinetics (PK) trial with fusidic acid (which utilized a loading dose of 2200 mg and maintenance dose of 1100 mg or a 3300 mg loading dose and 1650 mg maintenance dose), the expected steady state trough concentrations of FA were 100 or 200 μg/mL, respectively [Still et al. 2011. “Pharmacokinetics and safety of single, multiple, and loading doses of fusidic acid in healthy subjects.” Clinical infectious diseases: an official publication of the Infectious Diseases Society of America. 52 Suppl 7:S504-512]. However, in several illustrative examples of co-treatment with FA and RIF, subjects had fusidic acid concentrations lower than anticipated. During the first week of therapy, all subjects in that treatment cohort had fusidic acid concentrations lower than anticipated from the prior PK observations [Still, et al. 2011]. At week 4 and week 6, blood levels of fusidic acid continued to decline (see FIG. 1) By week 6, fusidic acid concentrations were 50-80% lower than the previously observed concentration with similar dosing regimens. These observations suggest a surprising and substantial drug-drug interaction whereby rifampin lowers fusidic acid concentrations.

A representative example of this drug-drug interaction is illustrated in the plasma concentrations of subject 111-01 (data points labeled “a” in FIG. 1). At the time of hospital discharge on Day 5, this patient had a Cmax of 95.6 μg/mL and AUC(0-t) of 581 μg·h/mL, having received 1800 mg TDD of fusidic acid and 900 mg TDD of rifampin. Following hospital discharge, a dose adjustment to fusidic acid TDD of 1200 mg and rifampin TDD of 300 was made to manage GI tolerance. At the week 4 visit, this patient's Cmax and AUC(0-t) had decreased substantially, to 64.6 μg/mL and 292 μg·h/mL, respectively. At this point rifampin was discontinued due to elevated bilirubin and the subject continued on fusidic acid monotherapy. By Week 6, with the subject only taking fusidic acid at a TDD of 1200 mg, the fusidic acid plasma concentration levels surprisingly increased. Specifically, the Cmax and AUC (0-t) were 117 μg/mL and 658 μg·h/mL, respectively. This Cmax is in line with the steady state plasma concentrations observed with a similar dosing regimen in the Phase 1 PK trial, see TABLE A [Still, et al. 2011]. Antimicrobial therapy was successful in this patient.

TABLE A

| | Fusidic | | | | | | |
|---|---|---|---|---|---|---|---|
| | Acid | Rifampin | | | | | |
| | TDD | TDD | Cmax | Tmax | AUC(0-t) | DN* Cmax | DN* AUCt |
| Visit | (mg) | (mg) | (μg/mL) | (h) | (μg · h/mL) | (μg/mL)/mg | (μg · h/mL)/mg |
| Day 5 | 1800 | 900 | 95.6 | 2.45 | 581 | 0.106 | 0.646 |
| Week 4 | 900 | 300 | 64.6 | 1.17 | 292 | 0.108 | 0.487 |
| Week 6 | 900 | 0 | 117 | 6 | 658 | 0.195 | 1.100 |

Each publication cited herein is incorporated herein by reference:

1. A method for treating a bone or joint infection, the method comprising administering fusidic acid or a salt thereof, and administering one or more agents selected from the group consisting of lipopeptide antibiotics, glycopeptides antibiotics, lipoglycopeptide antibiotics, oxazolidinone antibiotics, beta-lactam antibiotics, and combinations thereof.

2. A kit or package comprising a dose of fusidic acid or a salt thereof; and a dose of one or more agents selected from the group consisting of lipopeptide antibiotics, glycopeptides antibiotics, lipoglycopeptide antibiotics, oxazolidinone antibiotics, beta-lactam antibiotics, and combinations thereof; and a set of instructions for using the dose of fusidic acid or salt thereof and each dose of the one or more agents.

3. The method of claim 1 wherein the bone or joint infection is a prosthetic joint infection.

4. The method of claim 1 wherein the first administration of fusidic acid or salt thereof and the administration of at least one of the one or more agents is performed on the same day.

5. The method of claim 1 further comprising the step of discontinuing the administration of the one or more agents after a predetermined period of time, and continuing the administration of fusidic acid or salt thereof.

6. The method of claim 1 wherein the fusidic acid or salt thereof is administered on one day at a dose of at least about 3000 mg/day.

7. The method of claim 1 wherein the fusidic acid or salt thereof is administered on one day at a dose greater than 1200 mg/day and on subsequent days at a dose of at least about 1200 mg/day.

8. The method of claim 1 wherein at least one agent is a lipopeptide antibiotic, a glycopeptide antibiotic, or a lipoglycopeptide antibiotic.

9.-10. (canceled)

11. The method of claim 1 wherein at least one agent is an oxazolidinone antibiotic.

12. The method of claim 1 wherein at least one agent is a beta-lactam antibiotic.

13. (canceled)

14. The method of claim 1 wherein the agent is oritavancin or a salt thereof.

15. The method of claim 1 wherein the agent is dalbavancin or a salt thereof.

16. The method of claim 1 wherein the agent is vancomycin or a salt thereof.

17. The method of claim 1 wherein the agent is daptomycin or a salt thereof.

18. The method of claim 1 wherein the agent is telavancin or a salt thereof.

19. The method of claim 1 wherein the agent is linezolid or a salt thereof.

20. The method of claim 1 wherein the agent is tedizolid or a salt thereof.

21. The method of claim 1 wherein the agent is nafcillin or a salt thereof.

22. The method of claim 1 wherein the agent is cefazolin or a salt thereof.

23. The method of claim 1 wherein the agent is ceftriaxone or a salt thereof.
